(19) United States
(12) Patent Application Publication
(10) Pub. No.: US 2011/0171134 A1
(43) Pub. Date: Jul. 14, 2011

(54) (6S)-5-METHYLTETRAHYDROFOLIC ACID FOR THERAPY OF TISSUE INJURY

(76) Inventors: Bermans J. Iskandar, Madison, WI (US); Kirk J. Hogan, Madison, WI (US)

(21) Appl. No.: 13/001,381

(22) PCT Filed: Jun. 24, 2009
(86) PCT No.: PCT/US09/48508

§ 371 Date: Mar. 21, 2011

Related U.S. Application Data
(60) Provisional application No. 61/075,650, filed on Jun. 25, 2008.

(51) Int. Cl. A61K 49/00; C12Q 1/02

(52) U.S. Cl. 424/9.2; 435/29

(57) ABSTRACT

The present invention relates generally to the field of methods useful in treating tissue injury. More specifically, the present invention relates to (6S)-5-methyltetrahydrofolic acid compounds, and methods of using the (6S)-5-methyltetrahydrofolic acid compounds, to treat conditions associated with nervous system injury.

### FIELD OF THE INVENTION

The present invention relates generally to the field of compositions and methods useful in treating tissue injury. More specifically, the present invention relates to (6S)-5-methyltetrahydrofolic acid compounds, and methods of using the (6S)-5-methyltetrahydrofolic acid compounds, to treat conditions associated with nervous system injury.

### BACKGROUND OF THE INVENTION

Nervous system injuries afflict millions of people worldwide, including accidental injuries, for example, in automobile accidents, intentional injuries, for example, in crime or in battle, and unintentional injuries, for example, during surgical procedures within and surrounding the nervous system. Patients suffering nervous system injuries often experience mild to profound disability and pain due to the condition. A patient's ability to work, participate in self-care, move, sense one's surroundings, sleep, or perform the routine activities of daily life may be affected by nervous system injuries to varying degrees. Severe nervous system injuries may be fatal in acute, subacute and chronic timeframes arising from complications in other organ systems, for example, the cardiovascular system, the pulmonary system, the hematologic system, the immune system, the gastrointestinal system, the urologic system, or the musculoskeletal system. In addition to physical ailments, nervous system injuries often have detrimental effects on a patient's psychological well-being causing, for example, social isolation and emotional depression.

Current methods for treating nervous system injuries suffer from a number of drawbacks. Most contemporary treatments focus on prevention of ongoing injury, for example, spinal column stabilization, prevention of tissue swelling, and anticoagulation to prevent venous thrombosis, and management of consequent signs and symptoms, for example, artificial ventilation, fluid and pain management, and prevention of infection. Many of these interventions carry significant risks. None of these or other contemporary treatments specifically address the underlying problem of regeneration, healing and recovery of nervous system tissues. Accordingly, the need exists for new compositions and methods that are effective in treating nervous system injuries.

### SUMMARY OF THE INVENTION

The present invention relates generally to the field of compositions and methods useful in treating tissue injury. More specifically, the present invention relates to (6S)-5-methyltetrahydrofolic acid compounds, and methods of using the (6S)-5-methyltetrahydrofolic acid compounds, to treat conditions associated with nervous system injury.

In some embodiments, the present invention provides a method of therapy for nervous system injury, comprising administering an effective amount of (6S)-5-methyltetrahydrofolic acid to a subject with nervous system injury, wherein said administering comprises therapy for said nervous system injury in said subject. In other embodiments, the nervous system injury is central nervous system injury. In other embodiments, the nervous system injury is spinal cord injury. In further embodiments, the nervous system injury is cranial nerve injury. In still further embodiments, the nervous system injury is peripheral nerve injury. In yet further embodiments, the nervous system injury is autonomic nervous system injury.

In some embodiments of the present invention, the injury is traumatic injury. In other embodiments, the traumatic injury is sharp traumatic injury. In further embodiments, the sharp traumatic injury is iatrogenic traumatic injury. In some embodiments, the traumatic injury is blunt traumatic injury. In further embodiments, the blunt traumatic injury is iatrogenic traumatic injury.

In some embodiments of the present invention the therapy is therapy before the nervous system injury. In other embodiments, the therapy is therapy during the nervous system injury. In further embodiments, the therapy is therapy after the nervous system injury.

In some embodiments of the present invention, the subject is a mammal. In preferred embodiments, the mammal is a human. In further embodiments, the subject is a surgical subject. In yet further embodiments, the subject is an anesthetic subject. In some embodiments, the subject is a fetus, a neonate, an infant, a child, a young adult, an adult, or an elderly adult. In particular embodiments, the subject has a pre-existing disorder in S-adenosyl methionine synthesis. In some particular embodiments, the pre-existing disorder in S-adenosyl methionine synthesis is an inborn disorder, for example, a disorder of the genes encoding proteins that participate in folate, cobablamin, or methylation reactions. In further embodiments, the pre-existing disorder in S-adenosyl methionine synthesis is an acquired disorder, for example, a nutritional deficiency, a toxic exposure, a drug exposure (for example, methotrexate or nitrous oxide), or a disease (for example, pernicious anemia or amyotrophic lateral sclerosis).

In some embodiments of the present invention, the effective amount of (6S)-5-methyltetrahydrofolic acid is between 0.0025 mg/kg and 200 mg/kg. In other embodiments, the effective amount of (6S)-5-methyltetrahydrofolic acid is between 20 ug/kg and 800 ug/kg.

In other embodiments, the effective amount of (6S)-5-methyltetrahydrofolic acid is between 60 ug/kg and 90 ug/kg. In still other embodiments, the effective amount of (6S)-5-methyltetrahydrofolic acid is between 80 and 90 ug/kg.

In some embodiments of the present invention, the administering is enteral administering. In other embodiment, the administering is parenteral administering. In particular embodiments, the parenteral administering is intramuscular, subcutaneous, intraperitoneal, transdermal, intra-cerebrospinal fluid or intravenous administering. In preferred embodiments, the administering is serial administering.

In some embodiments of the present invention, the administering comprises administering an effective amount of at least one additional compound to a subject with nervous system injury, wherein the administering comprises therapy for said nervous system injury in said subject. In one embodiment, the additional compound is active in S-adenosyl methionine synthesis, for example, homocysteine. In other embodiments, the additional compound is active in a metabolic pathway mediated by a cofactor such as folate, cobalamin and pyridoxine. In further embodiments, the additional compound is a substrate in a metabolic pathway mediated by a cofactor selected from the group consisting of folate, cobalamin and pyridoxine, for example, betaine or choline. In still further embodiments, the additional compound is an agonist or antagonist of a pathway active in S-adenosyl methionine utilization, for example, PARP-1 inhibitors including, for example, 3-aminobenzamide, 1,5 dihydroxyisoquinolone, tricyclic benimidazoles (described, for example, in U.S. patent application Ser. No. 11/490,090, filed Jul. 21, 2006, incorporated by reference herein in its entirety), 4-Amino-1,8-naphthalimide, 3,4-dihydro-5-[4-(1-piperidinyl)butoxy]-1(2H)-isoquinolinone, temozolomide, CEP-6800, AG14361, tricyclic lactam indole, 2-arylbenzimidazole-4-carboxamide pharmacophores, NU1025, GPI 15427 (Guilford Pharmaceuticals, Baltimore Md.), INO-10001, GPI 6150, ISQ, DPA, PHT, INH2BP, PJ 34, ono-1924H, DR-2313, GPH, 5-AIQ, nicotinamide and minocycline, and, for example, DNMT activators, for example, 5′-S-(propionic acid)5′-deoxy-0-(1′-beta-d-ribofuranosyl)1,3-dideazaadenine. In other embodiments, the additional compound is an antioxidant, for example, N-acetylcysteine. In other embodiments, the additional compound is an inhibitor of glial scar formation, for example, antibodies against NG2 chondroitin sulfate proteoglycan.

In some embodiments of the present invention, the therapy is regeneration of said nervous system. In other embodiments, the regeneration is cellular regeneration of the nervous system. In other embodiments, the cellular regeneration is regeneration of neurons in the nervous system. In further embodiments, the therapy is recovery of nervous system function after nervous system injury.

In some embodiments, the present invention provides a method of therapy for nervous system injury, comprising administering an effective amount of a synthetic reduced folate to a subject with nervous system injury, wherein the administering comprises therapy for the nervous system injury in the subject.

In other embodiments, the present invention provides a method of therapy for nervous system injury, comprising administering an effective amount of a naturally occurring folate to a subject with nervous system injury, wherein the administering comprises therapy for the nervous system injury in the subject.

In some embodiments, the present invention provides a method of treating nervous system injury, comprising administering a therapeutically effective amount of (6S)-5-methyltetrahydrofolic acid to a subject in need thereof to ameliorate a symptom of the nervous system injury. In some embodiments, the administering comprises administering to the subject a therapeutic agent selected from the group consisting of a second folate-containing compound, a cobalt-containing compound, and an amino acid substrate. In preferred embodiments, the second folate-containing compound is folic acid, the cobalt-containing compound is cobalamin, and the amino acid substrate is methionine or S-adenosyl methionine.

In some embodiments, the present invention provides a method of promoting tissue regeneration after an injury, comprising parenterally administering an effective amount of a synthetic reduced folate to a subject with a tissue injury, wherein the administering promotes tissue regeneration in said tissue injury in said subject. In some embodiments, the tissue is nervous system tissue. In other embodiments, the nervous system tissue is neurons and/or glia. In particular embodiments, the nervous system tissue is adult nervous system tissue. In further embodiments, the nervous system tissue is nervous system stem cells. In preferred embodiments, the synthetic reduced folate is (6S)-5-methyltetrahydrofolic acid. In some embodiments, the injury is traumatic injury. In still further embodiments, the administering precedes the injury. In yet further embodiments, the administering is coincident with said injury. In particularly preferred embodiments, the administering follows the injury. In some embodiments, the administering further comprises administering an effective amount of one or more additional active compounds. In some embodiments, the additional compound is selected from the group consisting of betaine, dimethylglycine, sarcosine, methionine, S-adenosyl-methionine, choline serine, a compound comprising cobalt, and a compound comprising folate.

In some embodiments, the present invention provides a method for promoting tissue regeneration after an injury comprising: providing a subject with a tissue injury; providing an effective amount of a synthetic reduced folate; parenterally contacting said subject with said effective amount of a synthetic reduced folate; and detecting said regeneration, wherein said detecting is selected from the group consisting of molecular, biochemical, enzymatic, histologic, imaging, physiologic, and behavorial detecting. In further embodiments the tissue is nervous system tissue. In still further embodiments, the synthetic reduced folate is (6S)-5-methyltetrahydrofolic acid. In yet further embodiments, the subject has an inborn error of S-adenosyl-methionine metabolism.

### DESCRIPTION OF THE FIGURES

FIG. 1. shows a model used to evaluate spinal sensory axon regeneration into a peripheral nerve graft in vivo. FIG. 1. shows rat dorsal root ganglia (DRG) system after bilateral lesion of the dorsal columns of the spinal cord at a cervical level (C3) and introduction of a sciatic nerve graft. The nerve graft provides a supportive environment for axon growth. Removal of the nerve segment injures the peripheral axons of DRG neurons on one side (the “conditioning” lesion), whereas neurons on the opposite side are unaffected by the peripheral injury (“unconditioned”). At 2 weeks, a fluorescent tracer is injected 1 cm into the graft and is transported to the cell bodies of DRG neurons that are able to extend axons into the graft. The fluorescent tracer is taken up in the regenerated neurons only.

FIG. 2. shows that (6S)-5-methyltetrahydrofolic acid enhances regeneration of spinal axons into a peripheral nerve graft. Dorsal root ganglia (DRG) axons in the dorsal columns of the spinal cord were lesioned in adult rats treated with the indicated daily doses of (6S)-5-methyltetrahydrofolic acid and a segment of peripheral nerve implanted into the lesion site as in FIG. 1. After 2 weeks, axons regenerating through the graft were labeled with a retrograde tracer, and labeled DRG cell bodies were counted on the side opposite the peripheral nerve resection (right L4-6 DRGs in FIG. 1) (i.e., the unconditioned side). Eleven animals were used as no-(6S)-5-methyltetrahydrofolic acid treatment controls; the others were treated with intraperitoneal injections of (6S)-5-methyltetrahydrofolic acid (10, 20, 40, 60, 80, 120, 160, 180, 200, 400, 800 g/kg in 4, 4, 4, 3, 15, 3, 4, 4, 4, 4, and 4 animals, respectively) starting 3 days before the surgery and given daily until 2 weeks postoperatively. Measurable regeneration occurred only in the (6S)-5-methyltetrahydrofolic acid-treated animals. This dose-related response occurred in all treated animals except for the group receiving the 800 ug/kg dose.

FIG. 3. shows that the effects of (6S)-5-methyltetrahydrofolic acid on spinal axon regrowth are synergistic with the effects of a conditioning peripheral nerve injury. Adult rats were treated daily with the indicated doses of (6S)-5-methyltetrahydrofolic acid, and regeneration of spinal sensory axons into a peripheral nerve graft was measured as described in legend to FIG. 1. Axon regeneration by dorsal root ganglia (DRG) neurons subjected to a peripheral nerve lesion (left L4-6 DRGs in FIG. 1.) (i.e., the conditioned side) is shown. The conditioning lesion promoted regeneration of the spinal axons without (6S)-5-methyltetrahydrofolic acid administration (note the difference in scale for this graph compared to FIG. 2.). The addition of (6S)-5-methyltetrahydrofolic acid was synergistic with the peripheral nerve injury at enhancing the growth of the injured spinal axons (n=11, 4, 4, 4, 3, 15, 3, 4, 4, 4, 4, 4 for (6S)-5-methyltetrahydrofolic acid doses 0, 10, 20, 40, 60, 80, 120, 160, 180, 200, 400, 800 g/kg, respectively). The only exception was the highest dose tested of 800 g/kg, which resulted in decreased regeneration compared with the control animals.

### DETAILED DESCRIPTION OF THE INVENTION

### I. Definitions

To facilitate an understanding of the invention, the following terms have the meanings defined below.

As used herein, the term “subject” refers to organisms to be treated by the methods of the present invention. Such organisms preferably include, but are not limited to, mammals (e.g., murines, simians, equines, bovines, porcines, canines, felines, and the like), and most preferably includes humans. In the context of the invention, the term “subject” generally refers to an individual who will receive or who has received treatment (e.g., administration of a compound of the present invention and optionally one or more other agents) for a condition characterized by the dysregulation of apoptotic processes.

As used herein, the term “effective amount” refers to the amount of a compound (e.g., a compound of the present invention) sufficient to effect beneficial or desired results. An effective amount can be administered in one or more administrations, applications or dosages and is not limited intended to be limited to a particular formulation or administration route.

As used herein, the term “catheter” refers generally to a tube used for gaining access to a body cavity or blood vessel.

The term “pharmaceutical composition” refers to the combination of an active agent with a carrier, inert or active, making the composition especially suitable for diagnostic or therapeutic use in vivo or ex vivo.

The term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. [1975]).

The term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while sometime not in themselves pharmaceutically acceptable, may be employed in the preparation of salts useful as intermediates in obtaining the compounds of the invention and their pharmaceutically acceptable acid addition salts. Examples of bases include alkali metals (e.g., sodium) hydroxides, alkaline earth metals (e.g., magnesium), hydroxides, ammonia, and compounds of formula NW₄⁺, wherein W is C₁₋₄ alkyl, and the like. Examples of salts include, but are not limited to, acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the terms “solid phase supports” or “solid supports,” are used in their broadest sense to refer to a number of supports that are available and known to those of ordinary skill in the art. Solid phase supports include, but are not limited to, silica gels, resins, derivatized plastic films, glass beads, cotton, plastic beads, alumina gels, and the like. As used herein, “solid supports” also include synthetic antigen-presenting matrices, cells, liposomes, and the like. A suitable solid phase support may be selected on the basis of desired end use and suitability for various protocols. For example, for peptide synthesis, solid phase supports may refer to resins such as polystyrene (e.g., PAM-resin obtained from Bachem, Inc., Peninsula Laboratories, etc.), POLYHIPE) resin (obtained from Aminotech, Canada), polyamide resin (obtained from Peninsula Laboratories), polystyrene resin grafted with polyethylene glycol (TENTAGEL, Rapp Polymere, Tubingen, Germany) or polydimethylacrylamide resin (obtained from Milligen/Biosearch, California).

The term “sample” as used herein is used in its broadest sense. A sample suspected of indicating a condition characterized by injury may comprise a cell, tissue, or fluids, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

As used herein, the terms “purified”, or “to purify”, refer to the removal of undesired components from a sample. As used herein, the term “substantially purified” refers to molecules that are at least 60% free, preferably 75% free, and most preferably 90%, or more, free from other components with which they usually associated.

As used herein, the term “modulate” refers to the activity of a compound (e.g., a compound of the present invention) to affect (e.g., to promote or retard) an aspect of cellular function, including, but not limited to, cell growth, proliferation, apoptosis, and the like.

It will be noted that the structure of some of the compounds of the invention includes asymmetric carbon atoms. It is to be understood that the isomers arising from such asymmetry (e.g., all enantiomers and diastereomers) are included within the scope of the invention, unless indicated otherwise. Such isomers can be obtained in substantially pure form by classical separation techniques and by stereochemically controlled synthesis. Furthermore, the structures and other compounds and moieties discussed in this application also include all tautomers thereof. Alkenes can include either the E- or Z-geometry, where appropriate.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

As a general matter, compositions specifying a percentage are by weight unless otherwise specified. Further, if a variable is not accompanied by a definition, then the previous definition of the variable controls.

The term “EC₅₀” is art-recognized and refers to the concentration of a compound at which 50% of its maximal effect is observed.

As used herein, the term “toxic” refers to any detrimental or harmful effects on a cell or tissue as compared to the same cell or tissue prior to the administration of the toxicant.

The term “second agent” refers to a therapeutic agent other than the (6S)-5-methyltetrahydrofolic acid compounds in accordance with embodiments of the present invention.

The term “co-administration” refers to the administration of at least two agent(s) (e.g., (6S)-5-methyltetrahydrofolic acid) or therapies to a subject. In some embodiments, the co-administration of two or more agents/therapies is concurrent. In other embodiments, a first agent/therapy is administered prior to a second agent/therapy. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “combination therapy” includes the administration of a (6S)-5-methyltetrahydrofolic acid compound of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). “Combination therapy” may, but generally is not, intended to encompass the administration of two or more of these therapeutic agents as part of separate monotherapy regimens that incidentally and arbitrarily result in the combinations of the present invention. “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single injection having a fixed ratio of each therapeutic agent or in multiple, single injections for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection. The sequence in which the therapeutic agents are administered is not narrowly critical. “Combination therapy” also can embrace the administration of the therapeutic agents as described above in further combination with other biologically active ingredients and non-drug therapies (e.g., surgery or radiation treatment.) Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

### II. Therapeutic Applications of (6S)-5-Methyltetrahydrofolic Acid Compounds

Administration of the (6S)-5-methyltetrahydrofolic acid compounds of the present invention provide therapeutic benefits to patients suffering from various tissue injuries. In one aspect, the invention provides a method of treating a nervous system injury, comprising administering a therapeutically effective amount of a compound of (6S)-5-methyltetrahydrofolic acid to a subject in need thereof to ameliorate a symptom of the condition. In another aspect, the present invention provides a method of promoting regeneration of neurons in the adult nervous system, comprising exposing said cell to (6S)-5-methyltetrahydrofolic acid described herein.

Experiments conducted in the course of the development of embodiments of the present invention indicate that (6S)-5-methyltetrahydrofolic acid improves axonal growth and repair in the adult nervous system. (6S)-5-methyltetrahydrofolic acid is a reduced form of folate. Its bioavailability differs from the oxidized form i.e., folic acid. (6S)-5-methyltetrahydrofolic acid is able to cross the blood-brain barrier. Metafolin™, the formulation of (6S)-5-methyltetrahydrofolic acid used in the presently disclosed experimental examples, is a compound of Merck KGaA (U.S. Pat. Nos. 5,997,915 (filed Jan. 31, 1997), and 6,254,904 (filed Oct. 15, 1999)), each of which is incorporated by reference herein in their entireties. (6S)-5-methyltetrahydrofolic acid is synthesized by reduction of folic acid to tetrahydrofolate, leading to the formulation of a new chiral center and two diastereoisomers in equimolar ratio. In nature, methylfolate consists only of the pure L-isomer (6S-isomer). Merck KGaA has developed processes allowing the isolation of the natural L-methylfolate by selective crystallization. (6S)-5-methyltetrahydrofolic acid has been used for the dietary treatment and prevention of a variety of disorders including anemia, neural tube defects, cardiovascular disease, Alzheimer's disease, and cancer.

Use of (6S)-5-methyltetrahydrofolic acid for nervous system regeneration according to the present invention has many benefits. For example, unlike folic acid, (6S)-5-methyltetrahydrofolic acid does not require hepatic transformation by reduction and methylation which may be impaired by inborn and acquired disorders of the liver. As well, unmetabolized folic acid in excess of the upward limit of transformation may have inherent health risks, for example, decreased natural killer cell cytotoxicity. In particular, (6S)-5-methyltetrahydrofolic acid readily crosses cellular barriers including those blocking entry of small molecules into the central nervous system. Moreover, because (6S)-5-methyltetrahydrofolic acid is safe and non-toxic, it is marketed in the USA under Section 8 of the FDA Dietary Supplement Health and Education Act (DSHEA). As well, parenteral (6S)-5-methyltetrahydrofolic acid does not require gastrointestinal absorption, and distribution to the liver before reaching its cellular targets in the nervous system and other tissues.

In experiments conducted in the development of embodiments of the present invention it was demonstrated that (6S)-5-methyltetrahydrofolic acid influences repair mechanisms in the adult CNS, with a significant increase observed in the regeneration of axons into peripheral nerve grafts after damage to the spinal cord. (6S)-5-methyltetrahydrofolic acid exhibits a 30%, or greater, increase in regenerative efficacy when compared to approximately 16% increase with folic acid. As well, the slopes of the (6S)-5-methyltetrahydrofolic acid dose-effect curves are steeper than those seen with folic acid, with peak efficacy occurring in a narrower dose range. At no point in the (6S)-5-methyltetrahydrofolic acid dose-effect curves was toxicity observed. Rather, higher doses lead to a graduated loss of benefit, but not to specific toxicity indicative of apparent cell injury.

Surprisingly, the effect of (6S)-5-methyltetrahydrofolic acid on spinal axon regrowth is not equivalent to the effect of a peripheral injury. Rather, the influence of (6S)-5-methyltetrahydrofolic acid on axon regrowth is additive or synergistic with the neuronal cell body responses produced by peripheral nerve injury, which increase the intrinsic capacity of neurons for regeneration. Previous studies have suggested that the effects of peripheral nerve injury can be mimicked but were not augmented by the targeted expression of specific growth-associated genes, by infusion of appropriate neurotrophins, or by a transient elevation of cyclic AMP. While the present invention is not confined to a single mechanism and an understanding of the mechanism is not needed to practice the invention, experiments conducted in the course of development of embodiments of the present invention suggest that (6S)-5-methyltetrahydrofolic acid administration enhances regeneration of spinal sensory axons in vivo by an independent mechanism that acts synergistically with conditions known to promote axon regeneration through an effect on the neuronal cell body, and therefore potentially can be combined with these and other treatments to further improve axon repair.

In some embodiments, the present invention provides a method of treating post-operative cognitive dysfunction (POCD), comprising administering a therapeutically effective amount of (6S)-5-methyltetrahydrofolic acid to a subject in need thereof to ameliorate a sign or symptom of POCD. As used herein, POCD is the new onset after anesthesia and/or surgery of deficits in at least two areas of cognitive function lasting 2 weeks after surgery or longer. POCD is observed in 20% to 60% of patients after cardiopulmonary bypass (CPB) and in 10% to 16% of older patients following major, non-cardiac surgery. The elderly may be more susceptible to POCD. Surgery and perioperative interventions are associated with susceptibility to POCD. Risk factors implicated in the pathogenesis of POCD may include: advanced patient age; preoperative cognitive level of performance and education, the type of surgery (i.e., cardiac vs. non-cardiac), co-existing neurologic disease and the presence or absence of operative complications (e.g., post-operative respiratory infection), the number and frequency of operative procedures, micro-emboli (i.e., secondary to carotid endarterectomy, orthopedic procedures or new onset atrial fibrillation), hypotension, hypoxemia, anemia, electrolyte abnormalities (e.g., hyponatremia), genetic predispositions and altered glucose metabolism, among others. In some embodiments treating POCD comprises prevention of POCD by treatment before surgery and anesthesia. In other embodiments, treating POCD comprises prevention of POCD by treatment during surgery and anesthesia. In some embodiments, treatment of PCOD comprises treatment after surgery and anesthesia. In some embodiments, the administering comprises administering to the subject a therapeutic agent selected from the group consisting of a second folate-containing compound, a cobalt-containing compound, and an amino acid substrate. In preferred embodiments, the second folate-containing compound is folic acid, the cobalt-containing compound is cobalamin, and the amino acid substrate is methionine or S-adenosyl methionine.

In some embodiments, the present invention provides a method of treating tissue injury before, during and after surgery comprising administering a therapeutically effective amount of (6S)-5-methyltetrahydrofolic acid to a subject in need thereof. In other embodiments, the treating comprises promoting tissue repair and regeneration before, during and after surgery. As used herein the tissue may comprise, for example, integumentary tissue, cardiac tissue, vascular tissue, pulmonary tissue, skeletal muscle tissue, smooth muscle tissue, connective tissue, for example, ligament and tendon tissue, bone tissue, glandular tissue, gastrointestinal tissue, liver tissue, renal tissue, genito-urinary tissue, pancreas tissue, sensory tissue, for example, eye, ear, nose and mouth tissue, and dental tissue,

### III. Pharmaceutical Compositions, Formulations, and Exemplary Administration Routes and Dosing Considerations

Exemplary embodiments of various contemplated medicaments and pharmaceutical compositions are provided below. The invention is not limited to these illustrative examples.

### A. Preparing Medicaments

The compounds of the present invention are useful in the preparation of medicaments to treat or study a variety of tissue injuries. In certain embodiments, nervous system injury is associated with trauma. In other embodiments, nervous system injury is associated with, for example, infection, neoplasia, a heritable condition, a degenerative condition, thrombosis, ischemia, bleeding, embolus, intoxication, allergy, or aberrant immune response.

In addition, (6S)-5-methyltetrahydrofolic acid is also useful for preparing medicaments for treating or studying other nervous system conditions wherein the effectiveness of (6S)-5-methyltetrahydrofolic acid is known or predicted. The methods and techniques for preparing medicaments of (6S)-5-methyltetrahydrofolic acid of the present invention are well-known in the art. Exemplary pharmaceutical formulations and routes of delivery are described below.

### B. Exemplary Pharmaceutical Compositions and Formulation

In some embodiments of the present invention, (6S)-5-methyltetrahydrofolic acid is administered alone, while in some other embodiments, (6S)-5-methyltetrahydrofolic acid is preferably present in a pharmaceutical formulation comprising at least one active ingredient/agent, as discussed above, together with a solid support or alternatively, together with one or more pharmaceutically acceptable carriers and optionally other therapeutic agents. Each carrier should be “acceptable” in the sense that it is compatible with the other ingredients of the formulation and not injurious to the subject.

Contemplated formulations include those suitable for oral, rectal, nasal, topical (including transdermal, buccal and sublingual), vaginal, parenteral (including, for example, subcutaneous, intramuscular, intravenous, intraventricular, intracerebrospinal fluid, intraperitoneal, intra cardiac, and intradermal), and pulmonary administration. In some embodiments, formulations are presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient, for example, CerefolinNAC™; as a powder or granules; as a solution or suspension in an aqueous or non-aqueous liquid; or as an oil-in-water liquid emulsion or a water-in-oil liquid emulsion. In other embodiments, the active ingredient is presented as a bolus, electuary, or paste, etc.

In some embodiments, tablets comprise at least one active ingredient and optionally one or more accessory agents/carriers are made by compressing or molding the respective agents. In some embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention are optionally formulated as ointments, creams, suspensions, lotions, powders, solutions, pastes, gels, sprays, aerosols or oils. In alternative embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in a known manner. This phase typically comprises a lone emulsifier (otherwise known as an emulgent), it is also desirable in some embodiments for this phase to further comprise a mixture of at least one emulsifier with a fat or an oil or with both a fat and an oil.

Preferably, a hydrophilic emulsifier is included together with a lipophilic emulsifier so as to act as a stabilizer. In some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate.

Formulations suitable for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, and include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension including, for example, sodium carboxymethyl cellulose, sorbitol, and/or dextran. Optionally, the suspension may also contain stabilizers.

Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

### C. Exemplary Administration Routes and Dosing Considerations

Various delivery systems are known and can be used to administer (6S)-5-methyltetrahydrofolic acid e.g., encapsulation in liposomes, microparticles, microcapsules, receptor-mediated endocytosis, and the like. In specific embodiments, it may be desirable to administer (6S)-5-methyltetrahydrofolic acid locally to the area in need of treatment; this may be achieved by, for example, and not by way of limitation, local infusion during surgery, injection, or by means of a catheter, for example, into the cerebrospinal fluid. The compositions and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal, inhalational, aerosol, sublingual, intracardiac, intraventricular, spinal, epidural, regional or topical routes. It is also appreciated that the preferred route varies with the condition and age of the recipient, and the disease being treated.

Alternatively, or concurrently, administration may be by the oral route. The dosage administered will be dependent upon the age, health, and weight of the recipient, kind of concurrent treatment, if any, frequency of treatment, and the nature of the effect desired.

The agents identified can be administered to subjects or individuals susceptible to or at risk of failing to heal tissue injury. When the agent is administered to a subject such as a mouse, a rat or a human patient, the agent can be added to a pharmaceutically acceptable carrier and systemically or topically administered to the subject. To identify patients that can be beneficially treated, a tissue sample is removed from the patient and the cells are assayed for sensitivity to the agent.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. In other embodiments, the effective amount of (6S)-5-methyltetrahydrofolic acid is between 20 ug/kg and 800 ug/kg. In other embodiments, the effective amount of (6S)-5-methyltetrahydrofolic acid is between 60 ug/kg and 90 ug/kg. In still other embodiments, the effective amount of (6S)-5-methyltetrahydrofolic acid is between 80 and 90 ug/kg. When the compounds described herein are co-administered with another agent (e.g., a cobalt-containing compound or amino acid substrate), the effective amount may be less than when the agent is used alone.

Compositions within the scope of this invention include all compositions wherein the compositions of the present invention are contained in an amount which is effective to achieve its intended purpose. While individual needs vary, determination of optimal ranges of effective amounts of each component is within the skill of the art. Typically, the (65)-5-methyltetrahydrofolic acid may be administered to mammals, e.g. humans, orally or parenterally at a dose of 0.0025 to 200 mg/kg, for example, between 20 ug/kg and 800 ug/kg, between 60 ug/kg and 90 ug/kg, between 80 and 90 ug/kg, or an equivalent amount of the pharmaceutically acceptable salt thereof, per day of the body weight of the mammal being treated for disorders responsive to induction of regeneration. Preferably, about 0.01 to about 10 mg/kg is orally administered to treat, ameliorate, or prevent such nervous system injury. For example, the compositions may be administered at a dose of 5, 10, 20, 30, 40, 50, 60, 70, 80, 90, 100, 110, 120, 130, 140, 150, 160, 170, 180, 190, or 200 mg. For intramuscular injection, the dose is generally about one-half of the oral dose. For example, a suitable intramuscular dose would be about 0.000125 to about 25 mg/kg, and most preferably, from about 0.005 to about 5 mg/kg.

The unit oral dose may comprise from about 0.0025 to about 200 mg, preferably about 0.1 to about 500 mg of the composition, e.g., 1, 2, 3, 4, 5, 10, 15, 20, 25, 30, 35, 40, 45, 50, 55, 60, 65, 70, 75, 80, 85, 90, 95, 100, 150, 200, 250, 300, 350, 400, 450, or 500 mg. The unit dose may be administered one or more times daily or intermittently (e.g., once every 2, 3, 4, 5, 6, or 7 days or more) as one or more tablets or capsules each containing from about 0.1 to about 100 mg, conveniently about 0.25 to 50 mg of the composition, e.g., 1, 2, 3, 4, 5, 10, 15, 20, 25, 30, 35, 40, 45, 50, 55, 60, 65, 70, 75, 80, 85, 90, 95, or 100 mg.

In a topical formulation, the composition may be present at a concentration of about 0.0025 to 200 mg per gram of carrier. In a preferred embodiment, the composition is present at a concentration of about 0.07-1.0 mg/ml, more preferably, about 0.1-0.5 mg/ml, most preferably, about 0.4 mg/ml.

In addition to administering (6S)-5-methyltetrahydrofolic acid as a raw chemical, (6S)-5-methyltetrahydrofolic acid may be administered as part of a pharmaceutical preparation containing suitable pharmaceutically acceptable carriers comprising excipients and auxiliaries which facilitate processing of the compositions into preparations which can be used pharmaceutically. Preferably, the preparations, particularly those preparations which can be administered orally or topically and which can be used for the preferred type of administration, such as tablets, dragees, slow release lozenges and capsules, mouth rinses and mouth washes, gels, liquid suspensions, hair rinses, hair gels, shampoos and also preparations which can be administered rectally, such as suppositories, or by aerosol, as well as suitable solutions for administration by injection, topically or orally, contain from about 0.01 to 99 percent, preferably from about 0.25 to 75 percent of active compound(s), together with the excipient.

(6S)-5-methyltetrahydrofolic acid may be administered to any animal which may experience the beneficial effects of the compounds of the invention. Foremost among such animals are mammals, e.g., humans, although the invention is not intended to be so limited. Other animals include veterinary animals (cows, sheep, pigs, horses, dogs, cats and the like).

The pharmaceutical preparations of the present invention are manufactured in a manner which is itself known, for example, by means of conventional mixing, granulating, dragee-making, dissolving, or lyophilizing processes. Thus, pharmaceutical preparations for oral use can be obtained by combining (6S)-5-methyltetrahydrofolic acid with solid excipients, optionally grinding the resulting mixture and processing the mixture of granules, after adding suitable auxiliaries, if desired or necessary, to obtain tablets or dragee cores.

Compounds comprising (6S)-5-methyltetrahydrofolic acid may take the form of tablets, lozenges, granules, capsules, pills, ampoules, suppositories or aerosol form. They may also take the form of suspensions, solutions and emulsions of the active ingredient in aqueous or non-aqueous diluents, syrups, granulates or powders. In addition to an agent of the present invention, the pharmaceutical compositions can also contain other pharmaceutically active compounds or a plurality of compounds of the invention.

Ideally, the agent should be administered to achieve peak concentrations of the active compound at sites of disease. This may be achieved, for example, by the intravenous injection of the agent, optionally in saline, or by oral administration, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

### D. Exemplary Co-Administration Routes and Dosing Considerations

As described above, the invention includes methods involving co-administration of the compounds described herein with one or more additional active agents. Indeed, it is a further aspect of this invention to provide methods for enhancing prior art therapies and/or pharmaceutical compositions by co-administering a compound of this invention. In co-administration procedures, the agents may be administered concurrently or sequentially. In one embodiment, (6S)-5-methyltetrahydrofolic acid is administered prior to the other active agent(s). The pharmaceutical formulations and modes of administration may be any of those described above. The determination of appropriate type and dosage of radiation treatment is also within the skill in the art or can be determined with relative ease.

(6S)-5-methyltetrahydrofolic acid can be present in pharmaceutical compositions comprising a compound described herein and a pharmaceutically acceptable carrier. In certain embodiments, the pharmaceutical composition further comprises a second therapeutic agent. In certain embodiments, the second therapeutic agent is a cobalt-containing compound, for example, cobalamin. In other embodiments, the second therapeutic agent is a second folate compound. In further embodiments, the second therapeutic agent is an amino acid, for example methionine or S-adenosyl methionine. In instances where the known agent is toxic, it is desirable to limit the dosages administered in all cases, and particularly in those cases where drug resistance has increased the requisite dosage. When the compositions of the present invention are co-administered with the known agent, they reduce the dosage required which, in turn, reduces the deleterious effects.

### EXAMPLES

The invention, now being generally described, will be more readily understood by reference to the following examples, which are included merely for purposes of illustration of certain aspects and embodiments of the present invention, and are not intended to limit the invention.

In experiments conducted in the course of development of embodiments of the present invention it was shown that (6S)-5-methyltetrahydrofolic acid enhances the growth and repair mechanisms in the adult nervous system after injury. Using spinal cord lesion models of nervous system injury, it was observed that intraperitoneal treatment of adult rats with (6S)-5-methyltetrahydrofolic acid significantly improves the regrowth of sensory spinal axons into a grafted segment of peripheral nerve in vivo. These results demonstrate that the effects of (6S)-5-methyltetrahydrofolic acid administration on nervous system growth processes are not confined to the embryonic period, but are also effective for enhancing growth, repair and recovery in, for example, the injured adult nervous system.

### Experimental Example 1

### Spinal Cord Regeneration

### Materials and Methods

### Surgery

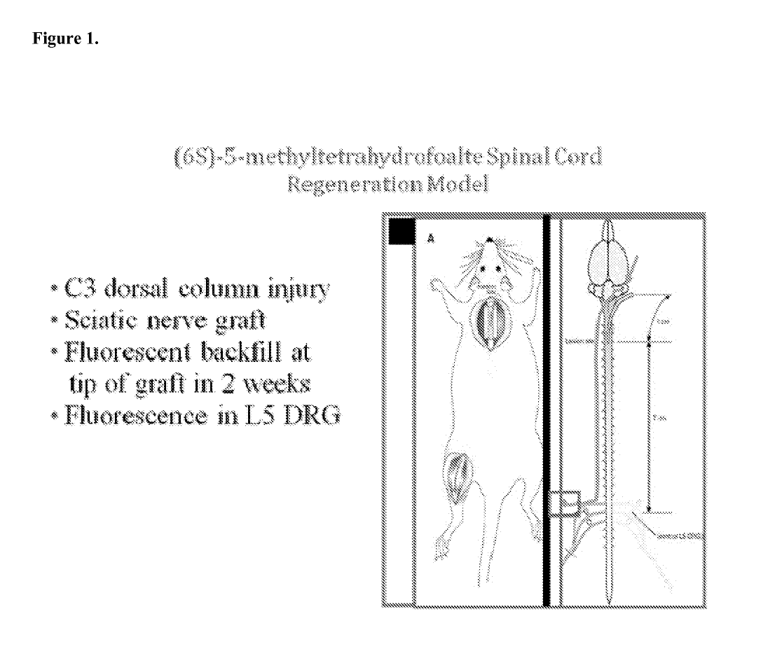

Sixty four adult male Sprague-Dawley rats (200-300 gm) were subjected to surgery under ketamine/xylazine anesthesia, as previously described (FIG. 1.) The cervical cord was exposed through a C3 laminectomy and dural opening. Using a pair of sharp jeweler's forceps, a well-defined 1 mm-deep injury was made in both posterior columns. A sciatic nerve segment harvested from the left hindlimb then was implanted at the injury site using 10-0 nylon sutures in the pia. The other end of the graft was left to lie freely under the skin.

Two weeks later, 5 μl of the retrograde tracer Fluorogold was placed into the nerve graft at a distance of 1 cm from the spinal cord. The fluorescent tracer does not diffuse down the graft and into the spinal cord. Rather, it is picked up solely by axons that have grown into the graft and is transported retrogradely in these axons.

### Perfusion and Tissue Preparation

Twenty four hours later, the animal was deeply anesthetized and perfused through the heart with 4% paraformaldehyde. The L5 dorsal root ganglia (DRG) were removed bilaterally, postfixed, incubated in 30% sucrose overnight, and quick frozen in Histo Prep (TM). Sections were cut at 10 μm with a cryostat, floated on pretreated glass slides, and stored at −80° C.

(6S)-5-Methyltetrahydrofolic acid Treatment

Animals were treated with intraperitoneal doses of (6S)-5-methyltetrahydrofolic acid ranging from 10 to 800 μg/kg, starting 3 days before the injury and given daily for 2 weeks.

### Data Analysis

Dorsal root ganglion sections were examined under a fluorescence microscope and the number of fluorescently labeled cells was counted (see FIG. 1.B). The same sections were then counterstained with cresyl violet to reveal the cell somas of all neurons in the DRG. Fluorescently labeled cells and total cells were counted in treated and control animals by an observer unaware of the treatment conditions. The percentage of labeled DRG cells in each animal was calculated. To determine neuronal numbers accurately, the method of Abercombie was used, in which only cells with visible nucleoli are counted.

### Statistical Analysis

The Wilcoxon rank-sum test and box-and-whiskers plots were used to compare the different groups.

### Results

### Spinal Cord Regeneration—Unconditioned Dorsal Root Ganglia

Without (6S)-5-methyltetrahydrofolic acid treatment, unconditioned DRG neurons

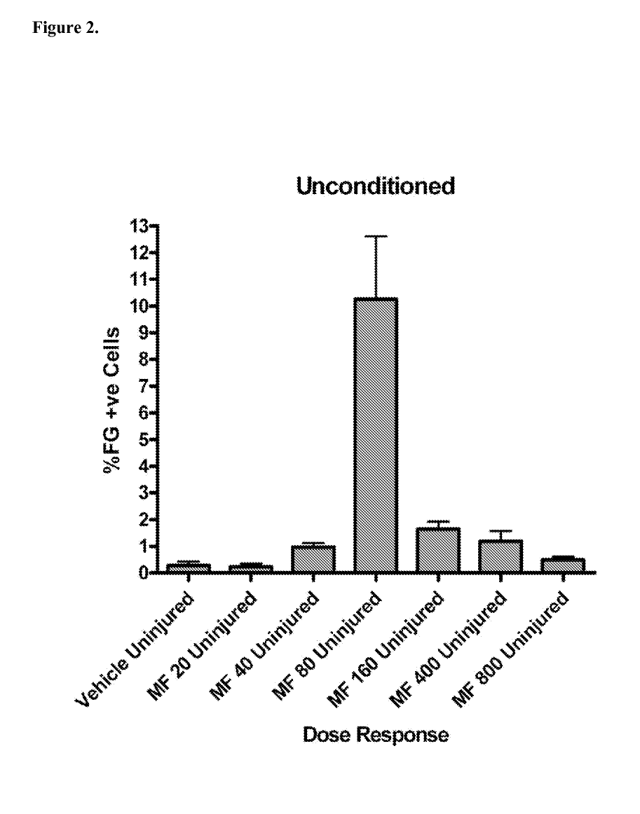

(contralateral to the peripheral injury) show no detectable fluorescent labeling, that is, no regeneration of their spinal axons into the graft (FIG. 2.). In contrast, animals treated with (6S)-5-methyltetrahydrofolic acid (80 N-g/kg) show a mean of 320 labeled neurons per ganglion (10.25%±2.30% SEM, n=13), in the absence of peripheral nerve injury (FIG. 2). The 80 ug/kg dose resulted in a statistically significant increase in the percentage of unconditioned neurons regenerating relative to all doses (p 0.05; one-way ANOVA with Bonferroni correction).

### Spinal Cord Regeneration—Conditioned Dorsal Root Ganglia

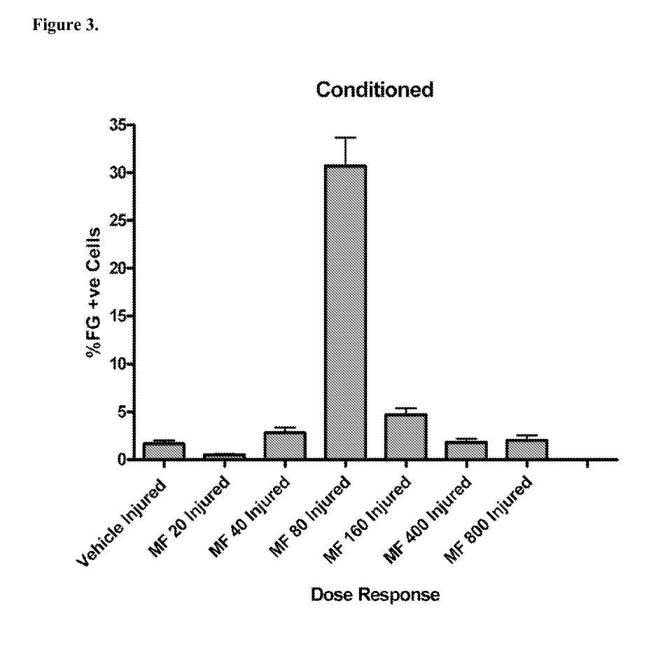

Daily injections of (6S)-5-methyltetrahydrofolic acid (80 ug/kg), starting 3 days before the injury and continuing for 2 weeks after the injury, produced a large increase in the percentage of conditioned DRG neurons that were fluorescently labeled (30.68%±2.97% SEM, n=13) in (6S)-5-methyltetrahydrofolic acid-treated animals, vs. 1.67%±0.31% SEM, n=13) in untreated controls). Higher or lower doses of (6S)-5-methyltetrahydrofolic acid were less effective, although regeneration was still greater than in control animals (FIG. 3.) All doses of (6S)-5-methyltetrahydrofolic acid from 0 to 800 ug/kg resulted in statistically significant increases in the percentage of conditioned neurons regenerating relative to the 800 ug/kg group (p≦0.05; one-way ANOVA with Bonferroni correction). The regeneration of injured spinal axons into a graft responds less to higher doses of (6S)-5-methyltetrahydrofolic acid (200-800 ug/kg). Although (6S)-5-methyltetrahydrofolic acid treatment without peripheral nerve injury (optimal dose of 80 ug/kg, see FIG. 2.), or peripheral nerve injury alone (no treatment group, see FIG. 3.) permits regeneration of approximately 319 and 53 axons per ganglion respectively, (6S)-5-methyltetrahydrofolic acid treatment combined with peripheral injury promotes regeneration by a mean of 980 neurons per ganglion (30.68%±2.97% SEM, n=13), a synergistic effect not previously observed with any other intervention. The responses did not change over the period of experimentation (15 months). These observations using (6S)-5-methyltetrahydrofolic acid compare favorably with 16.26%±1.12% SEM, (n=15) regeneration observed with folic acid. (Iskandar, Ann Neurol. 2004, August; 56(2):221-7.)

No neurological, behavioral, or hematological toxicity related to (6S)-5-methyltetrahydrofolic acid was observed in any of the groups tested at any dose. At the highest dose tested (800 ug/kg), there was no evidence of spinal regeneration in the unconditioned group, similar to the untreated control animals, and even decreased regeneration in the conditioned animals compared with controls.

### Experimental Example 2

### In Vitro Assays

### Materials and Methods

Three groups of adult male Sprague-Dawley rats (200-300 gm) are used for the in vitro assays: Group I, or uninjured animals (n=8); Group II, or animals with preceding spinal cord injury (n=8), in which a dorsal column injury is made as described above, but without placement of a sciatic nerve graft; and Group III, or animals with preceding sciatic nerve crush injury (n=16). Half of the animals in each group receive intraperitoneal injections of 80 μg/kg of (6S)-5-methyltetrahydrofolic acid daily for 3 days before injury. The control animals are euthanized 3 days after treatment with (6S)-5-methyltetrahydrofolic acid was started. Groups II and III animals are euthanized 48 hours after the injury (or 5 days after starting the (6S)-5-methyltetrahydrofolic acid injections).

### Dorsal Root Ganglia Culture

Immediately after euthanasia, the L4 and L5 ganglia are removed from the adult rats, dissociated, and centrifuged through a cushion of 10% Ficoll in F14 culture medium to remove myelinated axons, cellular debris, and nonneuronal cells as previously described. Neurons are resuspended in serum-free F14 medium containing N1 supplements and plated on polylysine/laminin coated glass coverslips. Cells from 12 to 14 ganglia are plated in a 24-well plate, and, after 18-24 hours, they are fixed in 4% paraformaldehyde for 30 minutes at room temperature.

### Visual Analysis of Neurite Growth

Quantitative analysis of neurite length and number of branch points per neurite are performed on fixed cultures displayed on a video monitor. Dilute cultures allow unambiguous measurements of neurites. The longest processes from each cell are chosen for measurement, and all counts are made by an observer unaware of the treatment conditions. The percentage of DRG neurons that extend processes longer than 300 μm are calculated in all groups of animals at various time intervals within a 48-hour period.

### Experimental Example 3

### Functional Recovery Model

### Materials and Methods

Adult male Sprague Dawley rats (200-300 gm) are anesthetized with halothane and undergo a T9 laminectomy under aseptic conditions. A 12.5 gm/cm injury is created using the NYU impactor._Rats received (6S)-5-methyltetrahydrofolic acid (80 μg/kg) in saline, or an equal volume of saline alone via an intraperitoneal injection every day for 2 weeks, beginning 3 days before the injury. The animals are videotaped for 4 minutes while ambulating in an open-field environment on the day after surgery, and weekly thereafter for a period of 6 weeks. Ambulatory function is scored in a blinded fashion using the BBB rating scale that assigns points for the frequency of occurrence of specific features of normal posture and locomotion. The scoring differences between (6S)-5-methyltetrahydrofolic acid and control groups are displayed in a graph illustrating the mean±standard error of the mean (SEM) at each time point. In addition, the primary end points of 6 weeks are analyzed using the Wilcoxon rank-sum test.

To assess the effects of folic acid on functional recovery from CNS damage in adult rats, the Basso, Beattie, and Bresnahan (BBB) scoring system is used to monitor neurological recovery from a standardized weight-drop contusion injury to the spinal cord. Subjects receive daily intraperitoneal injections of either (6S)-5-methyltetrahydrofolic acid (80 g/kg) or vehicle (saline), beginning 3 days before the injury and continuing for 2 weeks after injury. The subjects are then videotaped once a week for 6 weeks and scored by an observer unaware of the treatment conditions. With the contusion injury used, control animals recover voluntary locomotion, reaching a plateau at 2 to 3 weeks after spinal cord injury.

### Experimental Example 4

### Optic Nerve Regeneration

### Materials and Methods

### Surgery

Surgery is performed on adult male Sprague-Dawley rats. Control animals do not receive (6S)-5-methyltetrahydrofolic acid, and test animals receive daily (6S)-5-methyltetrahydrofolic acid injections started 3 days preoperatively and continued for 2 weeks postoperatively. The optic nerve is exposed through a lateral orbital approach and cut within 2 mm of the globe. One end of an autologous sciatic nerve graft is attached to the optic stump, while the distal end is left to lie freely under the skin. Two months later, 5 μl of the retrograde tracer Fluorogold is placed into the graft at a distance of 1.5 cm from the globe. Twenty four hours later, the animal is deeply anesthesized and perfused intracardially with 4% paraformaldehyde. The globe is removed and postfixed with 4% paraformaldehyde. The retina is dissected from the eye cup and examined under a fluorescence microscope.

### Statistical Analysis

An observer unaware of the treatment conditions records the number of retinal ganglion cells (RGCs) labeled with Fluorogold. The Wilcoxon rank-sum test is used to compare the two groups.

### REFERENCES

1. Rosenblatt D S, Fenton W A Inherited disorders of folate and cobalamin transport and metabolism. In: Scriver C R, Sly W S, Childs B, eds. The metabolic and molecular bases of inherited disease. 8 ed. New York: McGraw-Hill, 2000: 3897-3927.
2. Mattson M P, Shea T B. Folate and homocysteine metabolism in neural plasticity and neurodegenerative disorders. Trends Neurosci 2003; 26: 137-146.
3. Berry R J, Li Z, Erickson J D, et al. Prevention of neural-tube defects with folic acid in China. China-U.S. Collaborative Project for Neural Tube Defect Prevention. N Engl J Med 1999; 341: 1485-1490.
4. Czeizel A E, Dudas I. Prevention of the first occurrence of neural-tube defects by periconceptional vitamin supplementation. N Engl J Med 1992; 327: 1832-1835.
5. Martinasevic M K, Rios G R, Miller M W, Tephly T R. Folate and folate-dependent enzymes associated with rat CNS development. Dev Neurosci 1999; 21: 29-35.
6. Neumann S, Woolf C J. Regeneration of dorsal column fibers into and beyond the lesion site following adult spinal cord injury. Neuron 1999; 23: 83-91.
7. Bomze H M, Bulsara K R, Iskandar B J, et al. Spinal axon regeneration evoked by replacing two growth cone proteins in adult neurons. Nat Neurosci 2001; 4: 38-43.
8. Schnell L, Schwab M E. Axonal regeneration in the rat spinal cord produced by an antibody against myelin-associated neurite growth inhibitors. Nature 1990; 343: 269-272.
9. Richardson P M, McGuinness U M, Aguayo A J. Axons from CNS neurons regenerate into PNS grafts. Nature 1980; 284: 264-265.
10. David S, Aguayo A J. Axonal elongation into peripheral nervous system bridges after central nervous system injury in adult rats. Science 1981; 214: 931-933.
11. Benfey M, Aguayo A J. Extensive elongation of axons from rat brain into peripheral nerve grafts. Nature 1982; 296: 150-152.
12. Sawai H, Clarke D B, Kittlerova P, et al. Brain-derived neurotrophic factor and neurotrophin-4/5 stimulate growth of axonal branches from regenerating retinal ganglion cells. J Neurosci 1996; 16: 3887-3894.
13. Bregman B S, Kunkel-Bagden E, Schnell L, et al. Recovery from spinal cord injury mediated by antibodies to neurite growth inhibitors. Nature 1995; 378: 498-501.
14. Richardson P M, Issa V M. Peripheral injury enhances central regeneration of primary sensory neurones. Nature 1984; 309: 791-793.
15. Abercombie M. Estimation of nuclear population from microtome sections. Anat Rec 1946; 94: 239-247.
16. Coggeshall R E, La Forte R, Klein C M. Calibration of methods for determining numbers of dorsal root ganglion cells. J Neurosci Methods 1990; 35: 187-194.
17. Smith D S, Skene J H. A transcription-dependent switch controls competence of adult neurons for distinct modes of axon growth. J Neurosci 1997; 17: 646-658.
18. Gruner J. A monitored contusion model of spinal cord injury in the rat. J Neurotrauma 1992; 9: 123-128.
19. Basso D M, Beattie M S, Bresnahan J C. A sensitive and reliable locomotor rating scale for open field testing in rats. J Neurotrauma 1995; 12: 1-21.
20. Basso D M, Beattie M S, Bresnahan J C, et al. MASCIS evaluation of open field locomotor scores: effects of experience and teamwork on reliability. Multicenter Animal Spinal Cord Injury Study. J Neurotrauma 1996; 13: 343-359.
21. McDonald J W, Liu X Z, Qu Y, et al. Transplanted embryonic stem cells survive, differentiate and promote recovery in injured rat spinal cord. Nat Med 1999; 5: 1410-1412.
22. Bonilla I E, Tanabe K, Strittmatter S M. Small proline-rich repeat protein 1A is expressed by axotomized neurons and promotes axonal outgrowth. J Neurosci 2002; 22: 1303-1315.
23. Condic M L. Adult neuronal regeneration induced by transgenic integrin expression. J Neurosci 2001; 21: 4782-4788.
24. Ramer M S, Priestley J V, McMahon S B. Functional regeneration of sensory axons into the adult spinal cord. Nature 2000; 403: 312-316.
25. Cai D, Deng K, Mellado W, et al. Arginase I and polyamines act downstream from cyclic AMP in overcoming inhibition of axonal growth MAG and myelin in vitro. Neuron 2002; 35: 711-719.
26. Neumann S, Bradke F, Tessier-Lavigne M, Basbaum A I. Regeneration of sensory axons within the injured spinal cord induced by intraganglionic cAMP elevation. Neuron 2002; 34: 885-893.
27. Pasterkamp R J, Anderson P N, Verhaagen J. Peripheral nerve injury fails to induce growth of lesioned ascending dorsal column axons into spinal cord scar tissue expressing the axon repellent Semaphorin3A. Eur J Neurosci 2001; 13: 457-471.
28. Bradbury E J, Moon L D, Popat R J, et al. Chondroitinase ABC promotes functional recovery after spinal cord injury. Nature 2002; 416: 636-640.
29. Davies S J, Goucher D R, Doller C, Silver J. Robust regeneration of adult sensory axons in degenerating white matter of the adult rat spinal cord. J Neurosci 1999; 19: 5810-5822.
30. Kruman I I, Kumaravel T S, Lohani A, et al. Folic acid deficiency and homocysteine impair DNA repair in hippocampal neurons and sensitize them to amyloid toxicity in experimental models of Alzheimer's disease. J Neurosci 2002; 22: 1752-1762.
31. Kruman I I, Culmsee C, Chan S L, et al. Homocysteine elicits a DNA damage response in neurons that promotes apoptosis and hypersensitivity to excitotoxicity. J Neurosci 2000; 20: 6920-6926.
32. Lucock M. Folic acid: nutritional biochemistry, molecular biology, and role in disease processes. Mol Genet Metab 2000; 71: 121-138.
33. Hernandez-Diaz S, Werler M M, Walker A M, Mitchell A A. Folic acid antagonists during pregnancy and the risk of birth defects. N Engl J Med 2000; 343: 1608-1614.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1-58. (canceled)

59. A method for promoting nervous tissue regeneration after a nervous tissue injury comprising:
a) providing a subject with a nervous tissue injury;
b) providing an effective amount of a synthetic reduced folate;
c) contacting said subject with said effective amount of said synthetic reduced folate; and
d) detecting said nervous tissue regeneration, wherein said detecting is selected from the group consisting of molecular, biochemical, enzymatic, histologic, imaging, physiologic and functional detecting.

60. The method of claim 59, wherein said injury comprises central nervous system injury, spinal cord injury or cranial nerve injury.

61. The method of claim 59, wherein said injury is traumatic injury.

62. The method of claim 59, wherein said providing comprises providing before said injury, during said injury, after said injury, or a combination thereof.

63. The method of claim 59, wherein said subject is a mammal.

64. The method of claim 63, wherein said mammal is a human.

65. The method of claim 59, wherein said subject is a surgical subject.

66. The method of claim 59, wherein said synthetic reduced folate is (6S)-5-methyltetrahydrofolic acid.

67. The method of claim 66, wherein said effective amount of said (6S)-5-methyltetrahydrofolic acid is between 20 ug/kg and 800 ug/kg.

68. The method of claim 66, wherein said effective amount of said (6S)-5-methyltetrahydrofolic acid is between 60 ug/kg and 90 ug/kg.

69. The method of claim 66, wherein said effective amount of said (6S)-5-methyltetrahydrofolic acid is between 80 ug/kg and 90 ug/kg.

70. The method of claim 59, wherein said contacting is enteral contacting, parenteral contacting or serial contacting.

71. The method of claim 59, wherein said detecting said regeneration comprises regeneration of neurons, glia, or a combination thereof.
